(19) United States
(12) Patent Application Publication
(10) Pub. No.: US 2022/0346732 A1
(43) Pub. Date: Nov. 3, 2022

(54) DRIVING DEVICE FOR MOVING A TABLE

(71) Applicant: GE Precision Healthcare LLC, Wauwatosa, WI (US)

(72) Inventor: Akira Izuhara, Tokyo (JP)

(21) Appl. No.: 17/731,422

(22) Filed: Apr. 28, 2022

(30) Foreign Application Priority Data
Apr. 28, 2021 (JP) 2021-076888

(51) Int. Cl. A61B 6/04

(52) U.S. Cl. CPC A61B 6/0407; A61B 6/0487; A61B 6/035

(57) ABSTRACT

To provide technology that reduces vibrations in a yawing direction. A driving device 70 that drives a table 4 provided with a structure 50 including a cradle 40, comprising: a shaft 96 rotatable about a rotation axis along a longitudinal direction of the cradle 40; a first conversion part 100A provided on a first end side of the shaft 96 and that converts a rotational motion of the shaft 96 into a linear motion for moving the structure 50 in a transverse direction, where the transverse direction is a direction orthogonal to the longitudinal direction of the cradle 40 and a height direction of the table 4; and a second conversion part 100B provided on a second end side of the shaft 96 and that converts the rotational motion of the shaft 96 into a linear motion.

### CROSS-REFERENCE TO RELATED APPLICATIONS

This application claims priority to Japanese Patent Application No. 2021-076888, filed on Apr. 28, 2021, the disclosure of which is incorporated herein by reference in its entirety.

### BACKGROUND

The present invention relates to a driving device for moving a table in a transverse direction and a table including the drive device.

An X-ray computed tomography (CT) device is known as a medical device that non-invasively captures images of the inside of a specimen. X-ray CT devices can capture images of a site to be imaged in a short period of time, and therefore have become widespread in hospitals and other medical facilities.

The CT device has a gantry and a table. The table is configured to move in a z-direction such that a patient can be moved into a bore of the gantry. Furthermore, a type of CT device is known, which can also move the table in an x direction such that the x-direction (transverse direction) position of the site of a patient to be imaged relative to the bore can be adjusted to a position suitable for imaging.

FIG. 1 illustrates an example of a mechanism for moving a table 400 in an x-direction (transverse direction). FIG. 1 illustrates a plan view and side surface view of the table 400. The table 400 has a driving device 700 for moving a structure 500 of the table 400 in the x-direction (transverse direction). The driving device 700 is internally provided in the table 400. In the plan view, the driving device 700 is illustrated by a solid line to make the structure of the driving device 700 easier to visually recognize.

The driving device 700 has a ball screw 701. Rails 702 and 703 are provided on both sides of the ball screw 701. The rail 702 is slidably supported by two linear bearings 704 and 705 in the transverse direction, and the rail 703 is slidably supported by two linear bearings 706 and 707 in the transverse direction.

When the ball screw 701 rotates, the rotational motion is converted into a linear motion that moves the structure 500 in the transverse direction. Therefore, the rails 702 and 703 slide in the transverse direction in a condition supported by the linear bearings, such that the structure 500 can be moved in the transverse direction.

When the table 400 is moved in the transverse direction, it is important to suppress vibrations in a yawing direction 710 as much as possible such that image resolution is not reduced. Lengthening a span 711 between the two linear bearings is conceivable as a method of suppressing vibrations in the yawing direction 710. However, the span 711 is a value determined based on a table width 713 and a stroke 712 in the transverse direction, and a certain length of the stroke 712 must be ensured with regard to the table width 713. Therefore, there is a limit to how much the span 711 can be widened. Therefore, in the table 400 illustrated in FIG. 1, there is a problem where it is difficult to control vibrations in the yawing direction 710 when moving the table 400. In particular, when performing a cluster scan (non-helical scan), vibrations in the yawing direction 710 tends to appear, and there is a concern that the resolution of the image may decrease.

Therefore, a technology that can reduce vibrations in the yawing direction is desired.

### SUMMARY

A first aspect of the present invention is a driving device that drives a table provided with a structure including a cradle, including: a shaft rotatable about a rotation axis along a longitudinal direction of the cradle; a first conversion part provided on a first end side of the shaft and that converts a rotational motion of the shaft into a linear motion for moving the structure in a transverse direction, where the transverse direction is a direction orthogonal to the longitudinal direction of the cradle and a height direction of the table; and a second conversion part provided on a second end side of the shaft and that converts the rotational motion of the shaft into a linear motion.

A second aspect of the present invention is a table that moves an imaging subject, including: a structure that includes a cradle that supports the imaging subject; a shaft rotatable about a rotation axis along a longitudinal direction of the cradle; a first conversion part provided on a first end side of the shaft and that converts a rotational motion of the shaft into a linear motion for moving the structure in a transverse direction, where the transverse direction is a direction orthogonal to the longitudinal direction of the cradle and a height direction of the table; and a second conversion part provided on a second end side of the shaft and that converts the rotational motion of the shaft into a linear motion.

A third aspect of the present invention is a medical device having the table of the first aspect.

The present invention has two conversion parts. A rotational motion of the shaft is converted into a linear motion by two conversion parts disposed at intervals in a longitudinal direction of the shaft, and therefore, vibrations in the yawing direction of the table can be reduced.

### BRIEF DESCRIPTION OF THE DRAWINGS

FIG. 1 illustrates an example of a mechanism for moving a table 400 in an x direction (transverse direction).

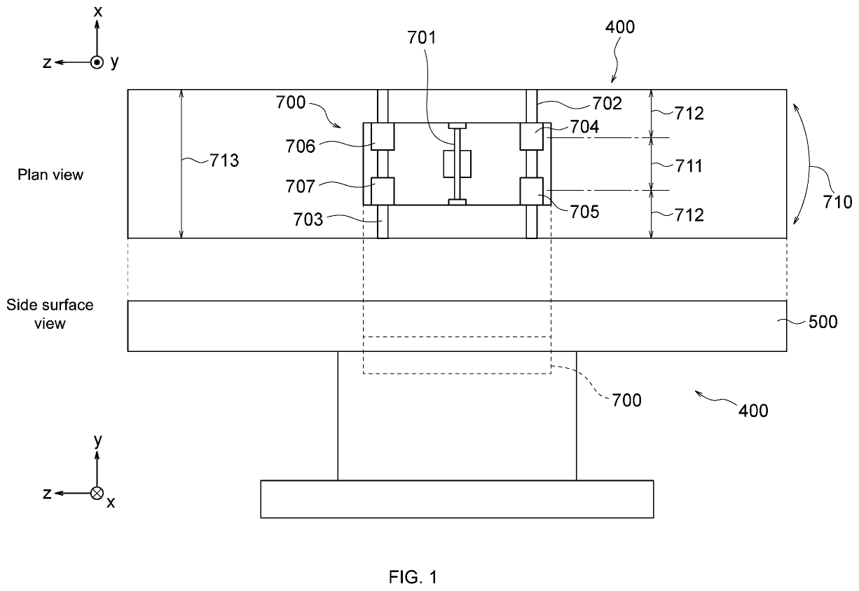

FIG. 2 is a perspective view of a CT device of an embodiment of the present invention.

FIG. 3 is a plan view and side surface view of a table 4.

FIG. 4 is a perspective view of a driving device 70.

FIG. 5 is a side surface view of the driving device 70 as viewed from a z direction.

FIG. 6 is a view where a frame 71 is separated from the driving device 70.

FIG. 7 is an exploded perspective view of the driving device 70.

FIG. 8 is a view illustrating a position of a rotating part 90.

FIG. 9 is a view illustrating securing positions of linear bearings 81A and 82A.

FIG. 10 is a view illustrating attaching positions of rails 83A and 83B.

FIG. 11 is an exploded perspective view illustrating a first conversion part 100A.

FIG. 12 is an exploded perspective view illustrating a belt driving part 800A.

FIG. 13 is a view illustrating an attaching position of a bracket 84A.

FIG. 14 is a view illustrating an attaching position of a drive pulley 85A.

FIG. 15 is a view illustrating attaching positions of idler pulleys 86A and 87A.

FIG. 16 is a view illustrating a securing position of a rack 88A.

FIG. 17 is an enlarged perspective view of the belt driving part 800A and a belt 89A.

FIG. 18 is a view illustrating the belt 89A attached to the belt driving part 800A.

FIG. 19 is an enlarged view of the first conversion part 100A.

FIG. 20 is a view illustrating an attaching position of a second conversion part 100B.

FIG. 21 is a perspective view of a driving device 70.

FIG. 22 is a side surface view of the driving device 70 as viewed from the z direction.

FIG. 23 is a perspective view illustrating the driving device 70 after moving a structure 50 in a −x direction.

FIG. 24 is a side surface view of the driving device 70 as viewed from the first conversion part 100A side.

FIG. 25 is a side surface view of the driving device 70 as viewed from the second conversion part 100B side.

FIG. 26 is a plan view and side surface view of the table 4 when the structure 50 is moved in the −x direction.

FIG. 27 is a perspective view illustrating the structure 50 moved by the driving device 70 in the x direction.

FIG. 28 is a plan view and side surface view of the table 4 when the structure 50 is moved in the x direction.

FIG. 29 is a view illustrating a table where a driving device 170 is provided on a base part 45.

### DETAILED DESCRIPTION

An embodiment for carrying out the invention will be described below, but the present invention is not limited to the following embodiment.

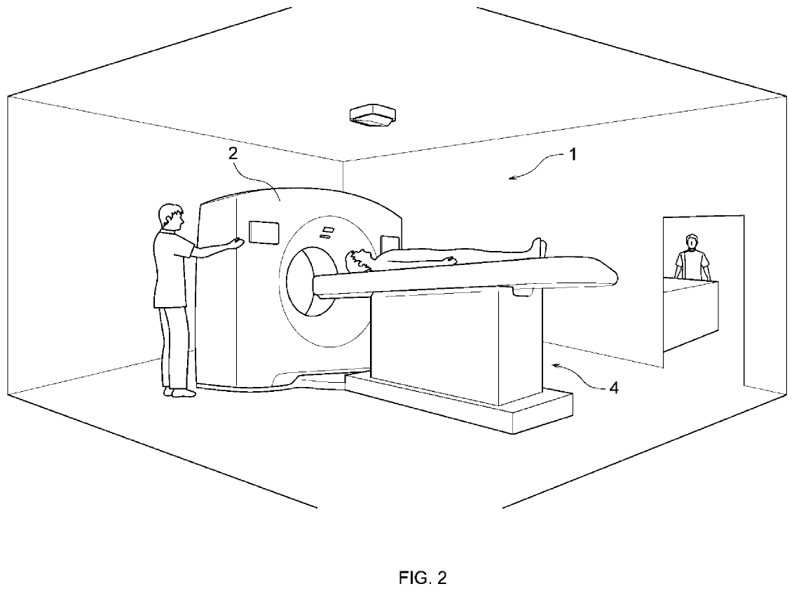

FIG. 2 is a perspective view of a CT device of an embodiment of the present invention. A CT device 1 has a gantry 2 and a table 4.

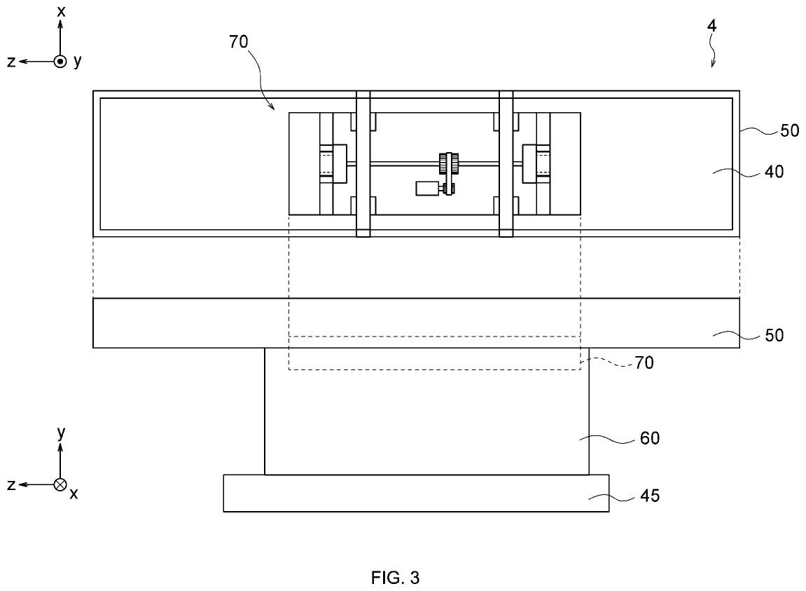

FIG. 3 is a plan view and side surface view of the table 4. In the plan view, a driving device 70 is illustrated by a solid line to make the structure of the driving device 70 easier to visually recognize. Note that herein, a longitudinal direction of a cradle 40 is a z direction, a height direction of the table 4 is a y direction, and a horizontal direction orthogonal to the z direction and y direction is an x direction.

The table 4 has a base part 45, an elevating part 60, a structure 50, and the driving device 70. The structure 50 includes the cradle 40 that supports a patient and a cradle supporting part that supports the cradle 40. The elevating part 60 supports the structure 50 such that the structure 50 can move in the height direction of the table (y direction, −y direction). The driving device 70 is provided inside the table 40. The driving device 70 drives the structure 50 such that the structure 50 moves in a transverse direction (x direction and −x direction).

In the present embodiment, the driving device 70 is configured to suppress vibrations in the yawing direction, which tend to occur when the structure 50 moves in the transverse direction. A structure of the driving device 70 will be described below.

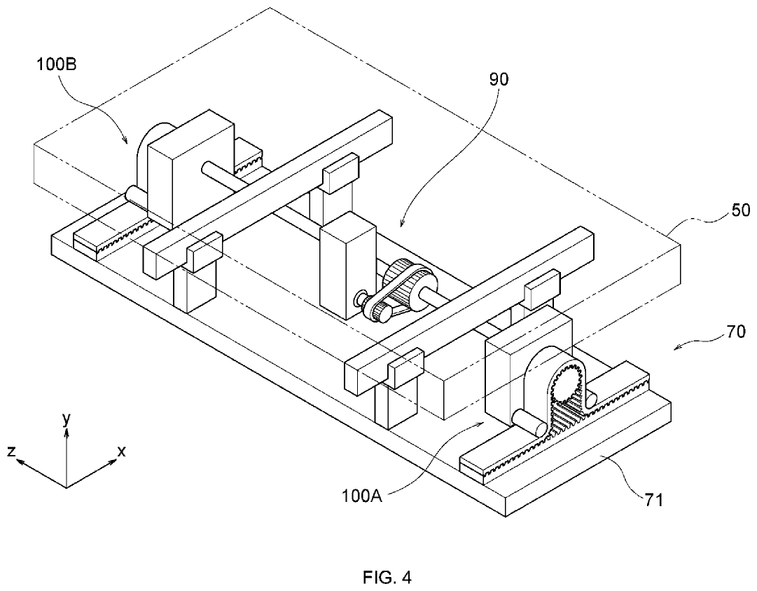

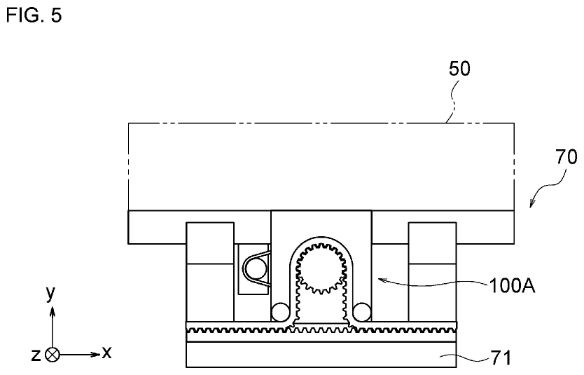

FIG. 4 is a perspective view of the driving device 70, and FIG. 5 is a side surface view of the driving device 70 as viewed from the z direction. The driving device 70 has, as primary components, a rotating part 90, a first conversion part 100A, and a second conversion part 100B. The first conversion part 100A and second conversion part 100B convert rotational motion of the rotating part 90 into linear motion for moving the structure 50 in the transverse direction. As a result, the structure 50 can be moved in the transverse direction.

Thus, the driving device 70 can drive the structure 50 such that the structure 50 moves in the transverse direction. Each component of the driving device 70 will be described in detail below.

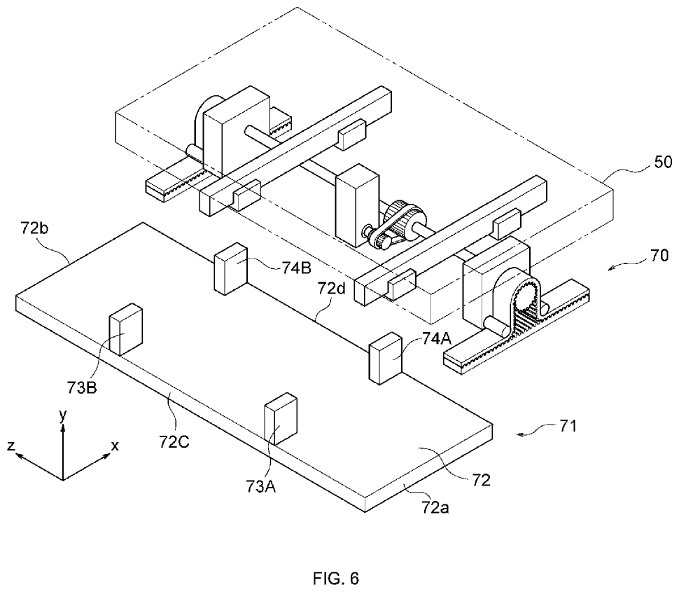

The driving device 70 has a frame 71. FIG. 6 is a view where the frame 71 is separated from the driving device 70.

The frame 71 has a frame main body 72 and pillar parts 73A, 74A, 73B, and 74B. The frame main body 72 is formed in a substantially flat plate shape.

The pillar parts 73A and 74A are formed at a position separated by a predetermined distance in the z direction from an end surface 72a of the frame main body 72. The pillar part 73A is formed on a side of an end surface 72c of the frame main body 72, and the pillar part 74A is formed on a side of an end surface 72d of the frame main body 72.

The pillar parts 73B and 74B are formed at a position separated by a predetermined distance in the −z direction from an end surface 72b of the frame main body 72. The pillar part 73B is formed on the side of the end surface 72c of the frame main body 72, and the pillar part 74B is formed on the side of the end surface 72d of the frame main body 72.

Various components of the driving device 70 are mounted on the frame 71 configured as described above. Components mounted on the frame 71 will be described below.

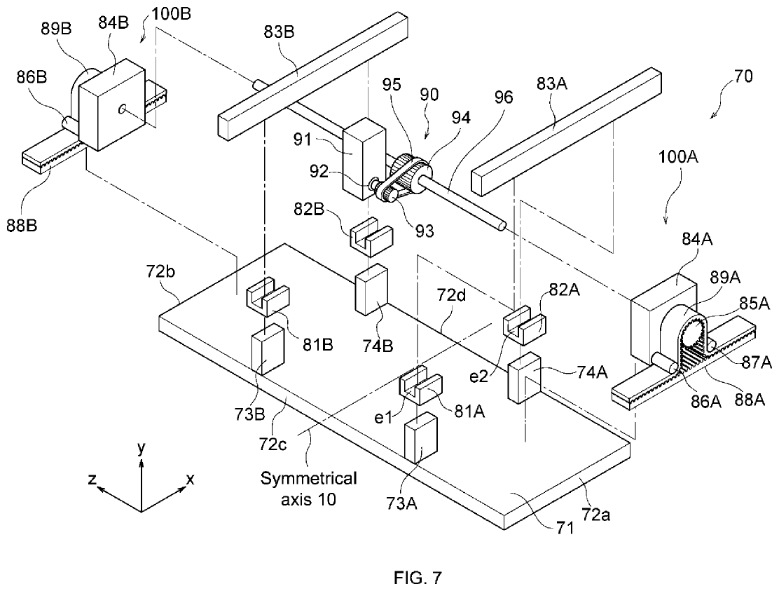

FIG. 7 is an exploded perspective view of the driving device 70. In FIG. 7, components denoted with a sign B and components denoted with a sign A have the same structure except for having mirror image relationships with regard to a symmetrical axis 10 (axis parallel to the x direction) that bisects the frame 71 in the z direction. Therefore, in describing each component of the driving device 70, the component marked with the sign A will be described in detail, and the components marked with the sign B will be briefly described.

The driving device 70 has the rotating part 90. The rotating part 90 has a base 91, a motor 92, a pulley 93, a pulley 94, a belt 95, and a shaft 96.

The motor 92 is secured inside the base 91. The pulley 93 is secured to a tip end of the motor 92. Furthermore, the shaft 96 is inserted through the other pulley 94. The shaft 96 is inserted through the pulley 94 along the z direction. The belt 95 is tensioned by the two pulleys 93 and 94.

When the motor 92 rotates, the belt 95 moves in a circulatory motion in a condition tensioned by the pulleys 93 and 94, such that the shaft 96 can rotate.

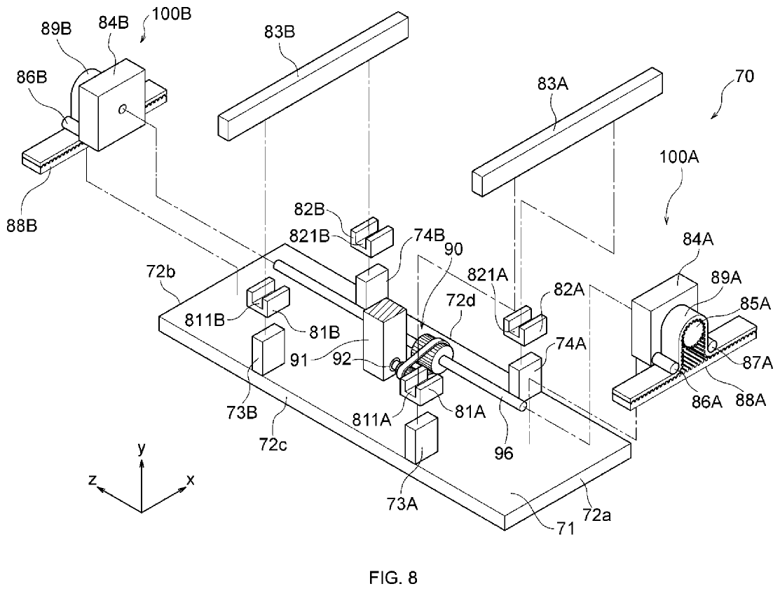

The rotating part 90 is provided such that the shaft 96 is positioned between the pillar parts 73A and 73B and the pillar parts 74A and 74B, as illustrated in FIG. 8. The shaft 96 extends along the z-axis. Note that an upper surface of the base 91 is secured to a bottom surface of the structure 50 (refer to FIG. 4). In FIG. 8, a portion of the base 91 that is secured to the structure 50 (refer to FIG. 4) is illustrated by diagonal lines to facilitate understanding that the upper surface of the base 91 is secured to the bottom surface of the structure 50.

Furthermore, the driving device 70 also has linear bearings 81A, 82A, 81B, and 82B.

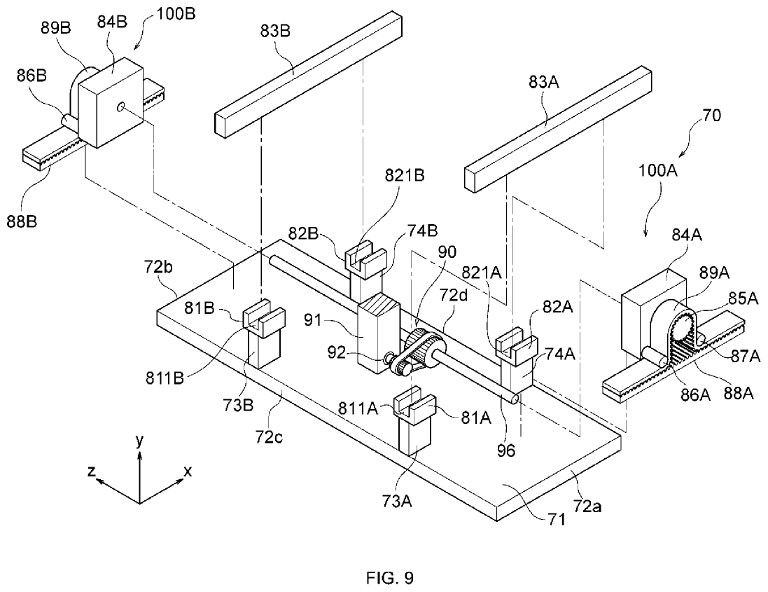

The linear bearings 81A and 82A have engaging parts 811A and 821A, respectively, which engage with a rail 83A, and the linear bearings 81B and 82B have engaging parts 811B and 821B, respectively, which engage with a rail 83B. As illustrated in FIG. 9, the linear bearings 81A and 82A are secured to upper surfaces of the pillar parts 73A and 74A of the frame 71, respectively. Furthermore, the linear bearings 81B and 82B are secured to upper surfaces of the pillar parts 73B and 74B of the frame 71, respectively.

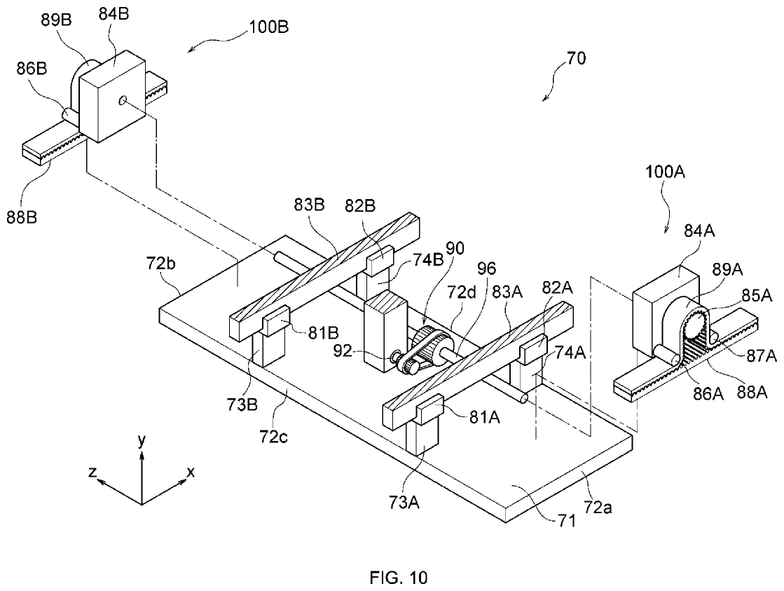

Furthermore, the driving device 70 has the rails 83A and 83B. As illustrated in FIG. 10, the rail 83A is slidably attached to the linear bearings 81A and 82A, and the rail 83B is slidably attached to the linear bearings 81B and 82B. The rails 83A and 83B extend in the x direction. Note that upper surfaces of the rails 83A and 83B are secured to the bottom surface of the structure 50 (refer to FIG. 4). In FIG. 10, portions of the rails 83A and 83B that are secured to the structure 50 (refer to FIG. 4) are illustrated by diagonal lines to facilitate understanding that the upper surfaces of the rails 83A and 83B are secured to the bottom surface of the structure 50. The rail 83B is secured to the structure 50 at a positioned separated in the longitudinal direction of the shaft 96 from the rail 83A.

Furthermore, the driving device 70 has the first conversion part 100A and the second conversion part 100B. The first conversion part 100A and second conversion part 100B convert rotational motion of the rotating part 90 into linear motion for moving the structure 50 in the transverse direction.

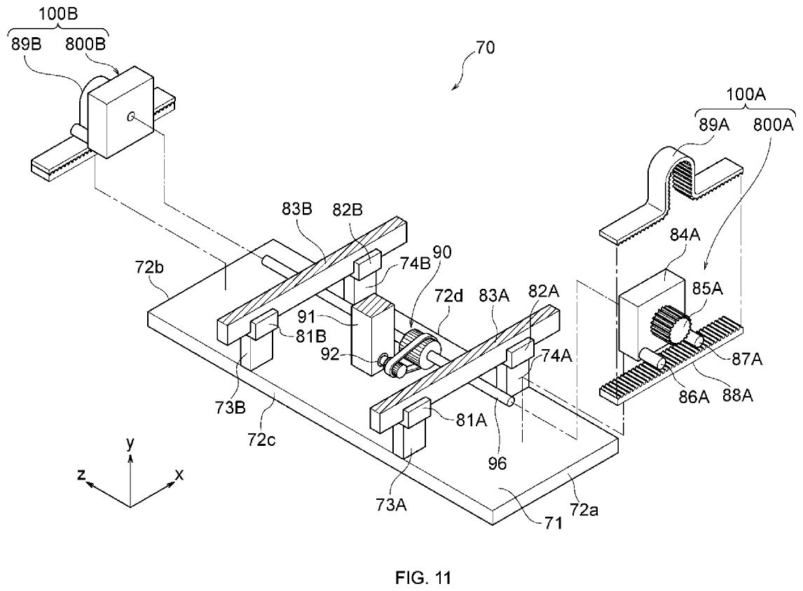

FIG. 11 is an exploded perspective view illustrating the first conversion part 100A.

The first conversion part 100A includes a belt 89A and a belt driving part 800A that drives the belt 89A.

The belt driving part 800A includes a bracket 84A, a drive pulley 85A, a pair of idler pulleys 86A and 87A, and a rack 88A. Each component of the belt driving part 800A will be described below.

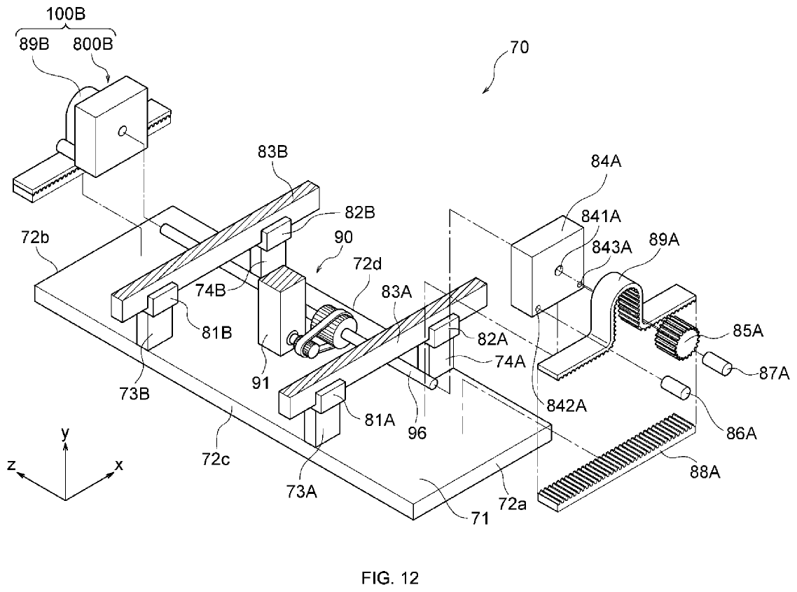

FIG. 12 is an exploded perspective view illustrating the belt driving part 800A. Through holes 841A, 842A, and 843A are formed in the bracket 84A. The through hole 841A is a hole through which the shaft 96 is inserted. The through hole 842A is a hole for axially supporting the idler pulley 86A, and the through hole 843A is a hole for axially supporting the idler pulley 87A.

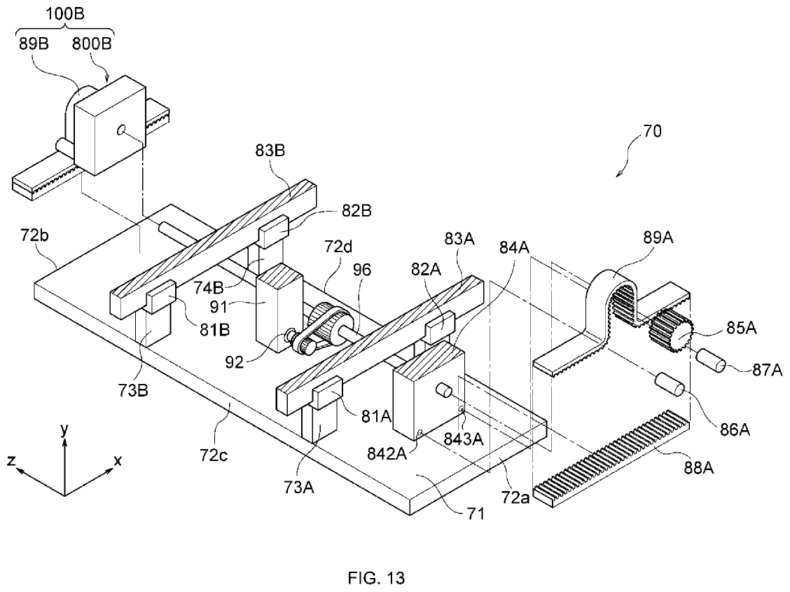

As illustrated in FIG. 13, the shaft 96 is inserted into the through hole 841A of the bracket 84A. The shaft 96 is axially supported by the bracket 84A through the through hole 841A. Note that an upper surface of the bracket 84A is secured to the bottom surface of the structure 50 (refer to FIG. 4). In FIG. 13, a portion of the bracket 84A that is secured to the structure 50 (refer to FIG. 4) is illustrated by diagonal lines to facilitate understanding that the upper surface of the bracket 84A is secured to the bottom surface of the structure 50.

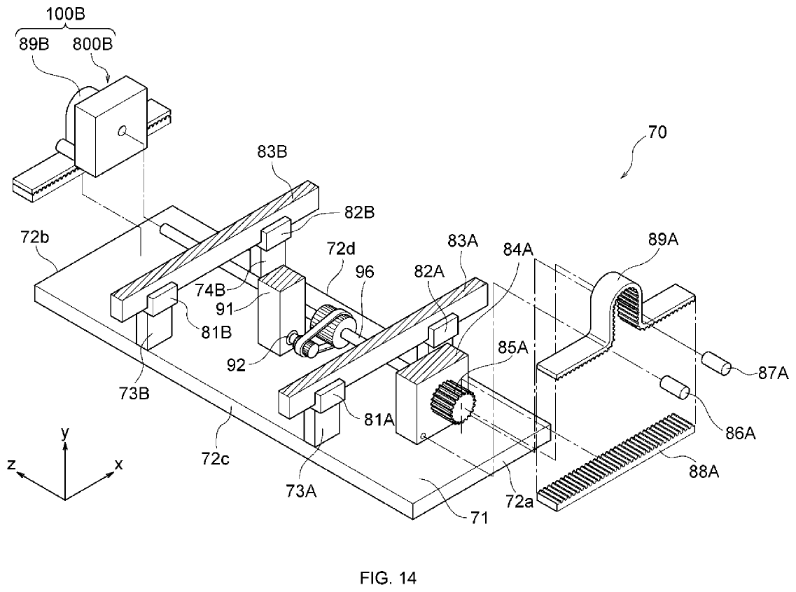

Furthermore, the drive pulley 85A is attached to a portion of the shaft 96 protruding from the bracket 84A, as illustrated in FIG. 14. The drive pulley 85A transmits the rotational motion of the shaft 96 to the belt 89A.

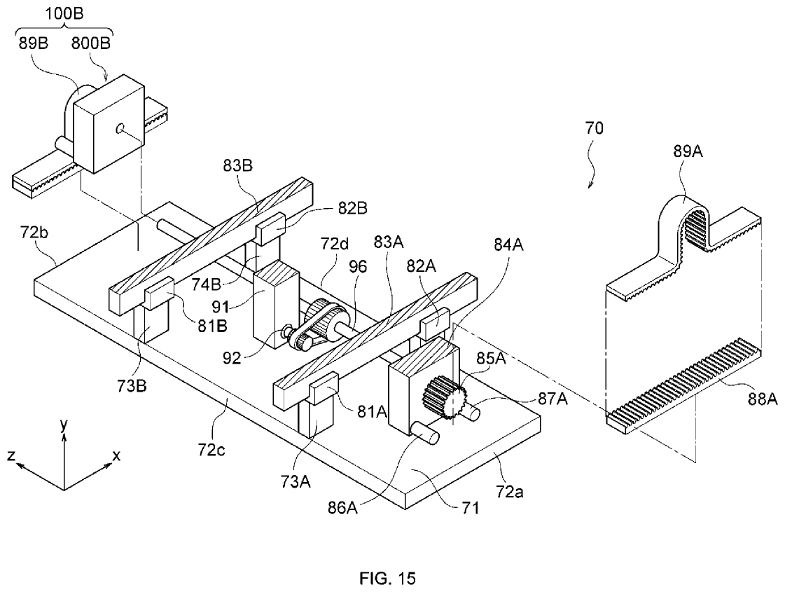

Furthermore, the pair of idler pulleys 86A and 87A, as illustrated in FIG. 15, are axially supported by through holes in the bracket 84A (through holes 842A and 843A illustrated in FIG. 13).

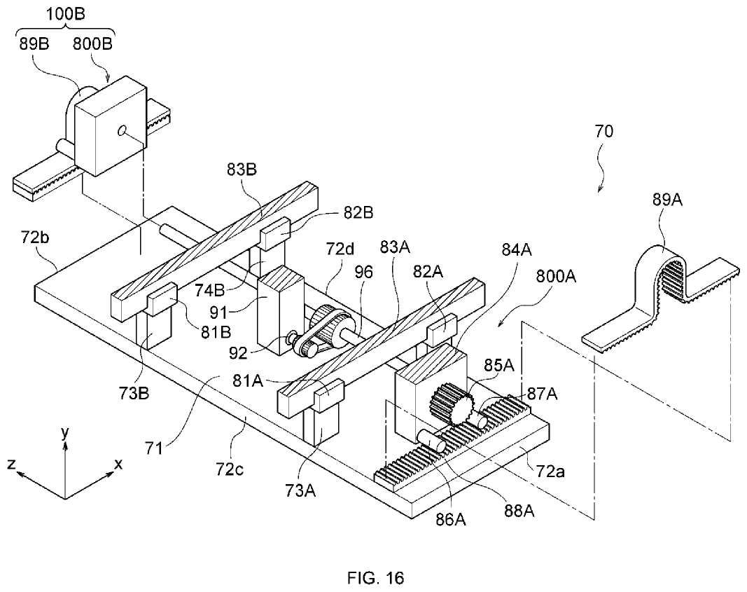

Furthermore, the rack 88A is secured to the frame 71, as illustrated in FIG. 16. The rack 88A extends in the x direction, passing under the idler pulleys 86A and 87A from the end surface 72c of the frame 71 to the end surface 72d on an opposite side. The belt driving part 800A is configured as described above.

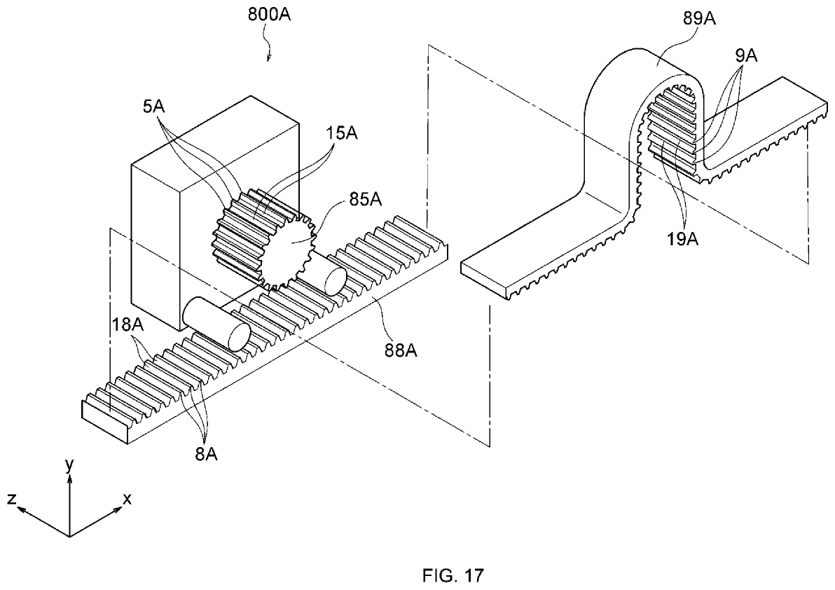

FIG. 17 is an enlarged perspective view of the belt driving part 800A and the belt 89A. A plurality of teeth 5A aligned in a rotational direction of the shaft 96 are formed on a surface of the drive pulley 85A. A groove 15A is formed between two adjacent teeth 5A.

A surface of the rack 88A has a plurality of teeth 8A aligned in the longitudinal direction (x direction) of the rack 88A. A groove 18A is formed between two mutually adjacent teeth 8A.

On the other hand, a plurality of teeth 9A aligned in the longitudinal direction of the belt 89A are formed on a back surface of the belt 89A. A groove 19A is formed between two mutually adjacent teeth 9A. The belt 89A configured in this manner is attached to the belt driving part 800A.

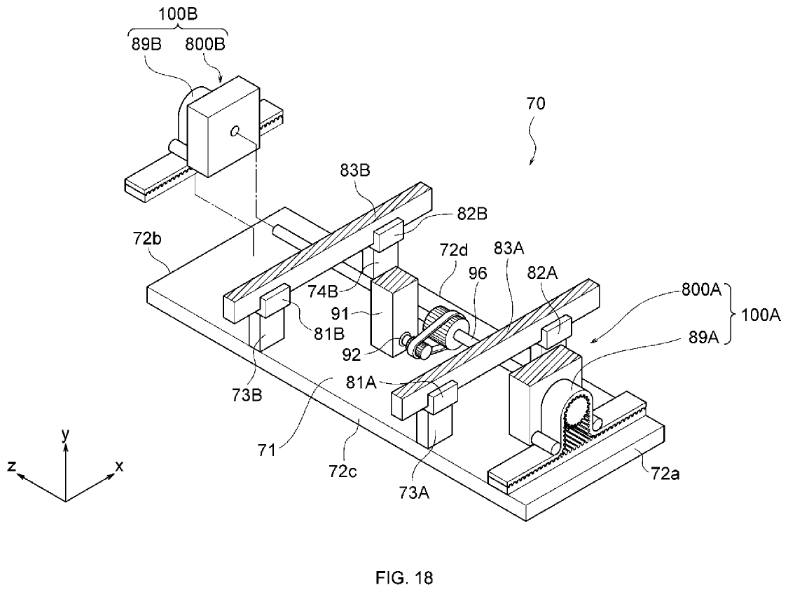

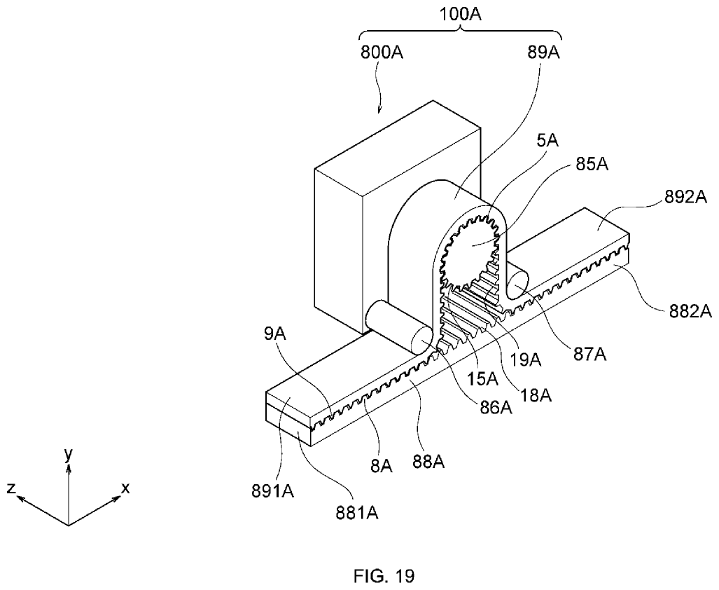

FIG. 18 is a view illustrating the belt 89A attached to the belt driving part 800A, and FIG. 19 is an enlarged view of the first conversion part 100A. The teeth 5A of the drive pulley 85A and the teeth 8A of the rack 88A mate with the groove 19A of the belt 89A. Furthermore, the teeth 9A of the belt 89A mate with the groove 15A of the drive pulley 85A and the groove 18A of the rack 88A.

Furthermore, end portions 891A and 892A of the belt 89A are secured to end portions 881A and 882A of the rack 88A, respectively, by a securing member (not illustrated), for example. The securing member can be a screw, for example.

The belt 89A is tensioned by the drive pulley 85A, the pair of idler pulleys 86A and 87A, and the rack 88A. Furthermore, the pair of idler pulleys 86A and 87A push the belt 89A against the rack 88A.

The idler pulley 86A presses the belt 89A against the rack 88A between the end portion 891A of the belt 89A and a portion of the belt 89A that is in contact with the drive pulley 85A. On the other hand, the idler pulley 87A presses the belt 89A against the rack 88A between the end portion 892A of the belt 89A and a portion of the belt 89A that is in contact with the drive pulley 85A. When the rotational motion of the shaft 96 is transmitted to the belt 89A by the drive pulley 85A, the rotational motion of the shaft can be converted into a linear motion to move the structure 50 in the transverse direction. The belt 89A is attached to the belt driving part 800A as described above.

The first conversion part 100A is configured as described above. As illustrated in FIG. 18, the first conversion part 100A is provided on a first end side of the shaft 96.

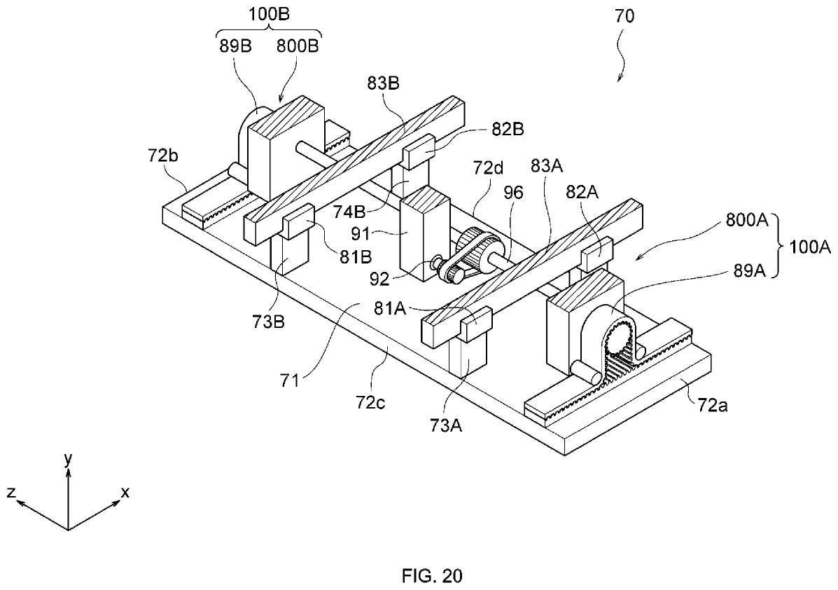

Furthermore, the driving device 70 also has the second conversion part 100B. The second conversion part 100B is provided on a second end side of the shaft 96, as illustrated in FIG. 20. The driving device 70 is configured as described above.

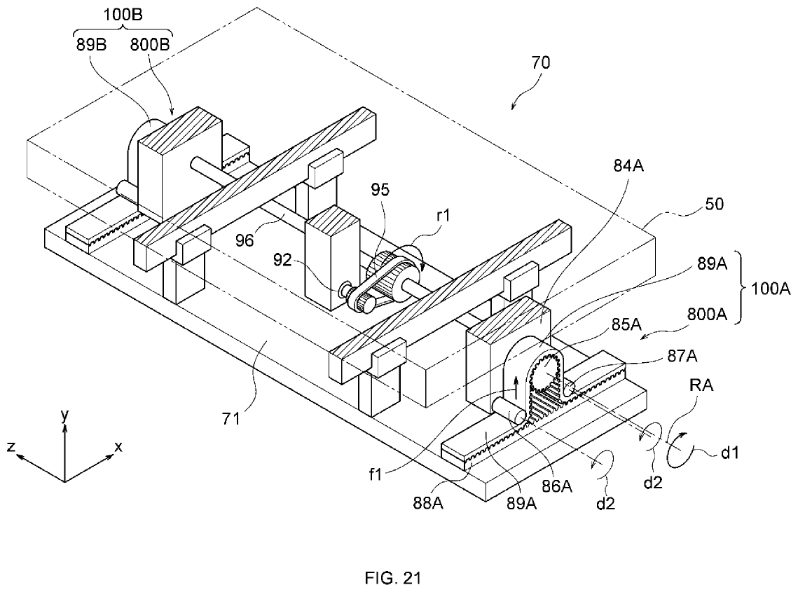

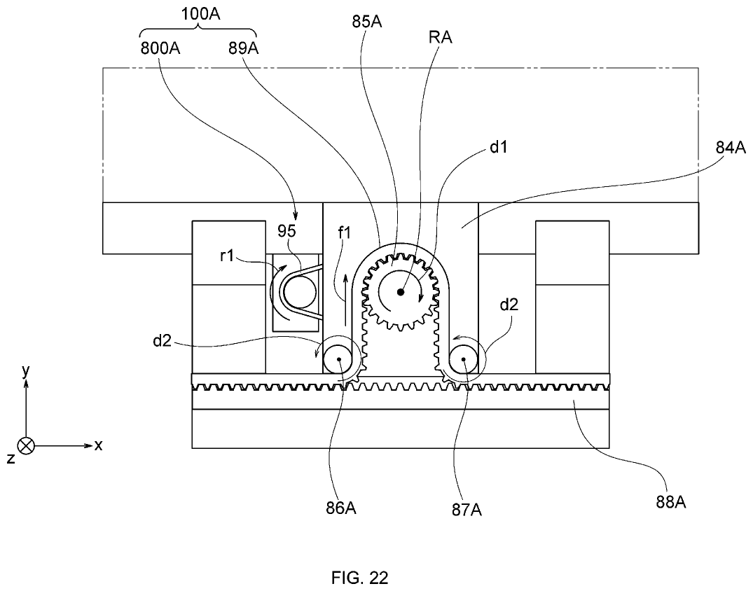

The driving device 70 can drive the structure 50 of the table 4 such that the structure moves in the transverse direction using the first conversion part 100A and second conversion part 100B. An operation of the driving device 70 will be described below with reference to FIG. 21, FIG. 22, and the like.

FIG. 21 is a perspective view of the driving device 70, and FIG. 22 is a side surface view of the driving device 70 as viewed from the z direction. First, the motor 92 is rotated to drive the belt 95. Herein, a case is considered where the belt 95 is driven such that the belt 95 moves in a circulatory motion in a direction r1. As the belt 95 moves in a circulatory motion in the direction r1, the shaft 96 rotates in a direction d1 about a rotation axis RA along the z direction.

The drive pulley 85A is attached to one end of the shaft 96. Therefore, the drive pulley 85A also rotates in the direction d1 about the rotation axis RA.

The idler pulley 86A pushes the belt 89A against the rack 88A. Therefore, when the drive pulley 85A rotates, a force f1 that pulls the belt 89A in the y direction is generated on the belt 89A. When the force f1 is generated, the idler pulley 86A moves in the −x direction while rotating in a direction d2.

Furthermore, the idler pulley 87A is axially supported by the bracket 84A. Therefore, the idler pulley 87A moves in the −x direction while rotating in the direction d2, similar to the idler pulley 86A. Therefore, the bracket 84A and the three pulleys 85A, 86A, and 87A move together in the −x direction. Note that even if the three pulleys 85A, 86A, and 87A move in the −x direction, there is no change in the relative positions of the three pulleys 85A, 86A, and 87A. Therefore, while the belt driving part 800A is driving the belt 89A, the belt 89A is still tensioned by the rack 88A and the three pulleys 85A, 86A, and 87A.

As described above, the belt driving part 800A drives the belt 89A, such that the rotational motion of the shaft 96 is converted into a linear motion form moving the structure 50 in the transverse direction (−x direction). Therefore, the first conversion part 100A can convert the rotational motion of the shaft 96 into a linear motion for moving the structure 50 in the transverse direction (−x direction).

Furthermore, the second conversion part 100B is provided on an opposite side of the first conversion part 100A from the shaft 96. The second conversion part 100B includes the belt 89B and the belt driving part 800B. Therefore, when the shaft 96 is rotated, the belt driving part 800B drives the belt 89B, such that the rotational motion of the shaft 96 is converted into a linear motion form moving the structure 50 in the transverse direction (−x direction). Therefore, the second conversion part 100B, similar to the first conversion part 100A, can convert the rotational motion of the shaft 96 into linear motion for moving the structure 50 in the −x direction.

Therefore, when the shaft 96 rotates, the structure 50 can be moved in the −x direction.

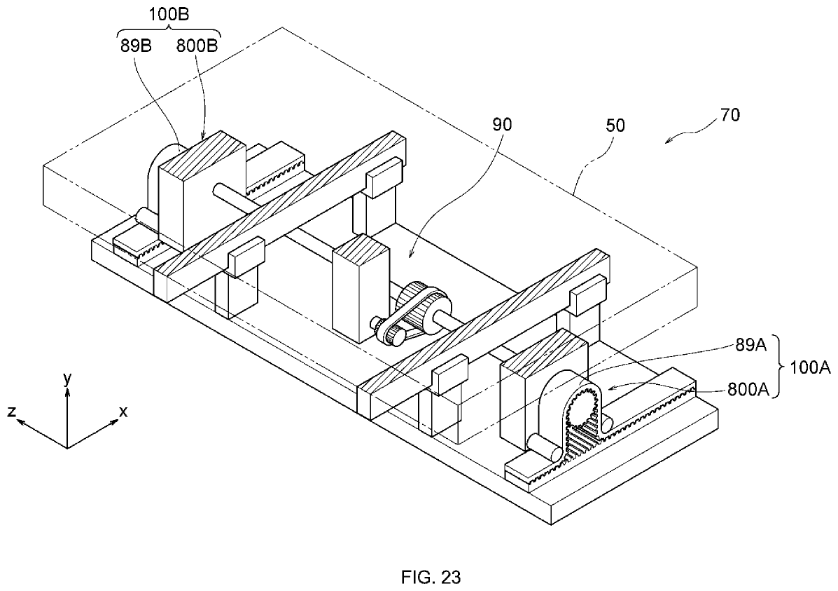

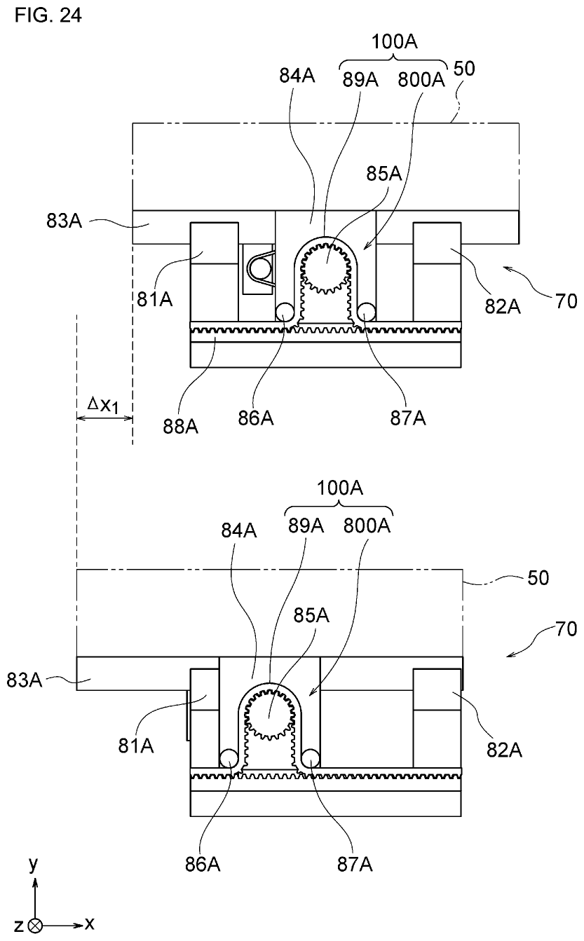

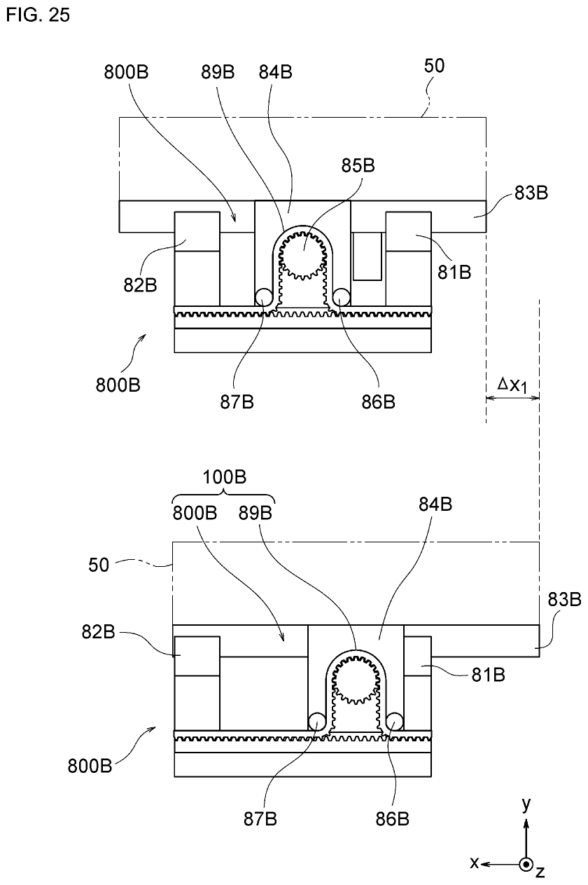

FIG. 23 is a perspective view illustrating the driving device 70 after moving the structure 50 in the −x direction. Furthermore, FIG. 24 is a side surface view of the driving device 70 as viewed from the first conversion part 100A side, and the FIG. 25 is a side surface view of the driving device 70 as viewed from the second conversion part 100B.

Note that the upper halves of FIG. 24 and FIG. 25 illustrate views before the driving device 70 moves the structure 50 in the −x direction, and the lower halves of FIG. 24 and FIG. 25 illustrate views after the driving device 70 moves the structure 50 in the −x direction.

As illustrated in FIG. 24, the three pulleys 85A, 86A, and 87A are attached to the bracket 84A. Therefore, when the belt driving part 800A drives the belt 89A, the bracket 84A and the three pulleys 85A, 86A, and 87A move together in the −x direction. FIG. 24 illustrates a movement of Δx1 in the −x direction.

Furthermore, as illustrated in FIG. 25, the three pulleys 85B, 86B, and 87B are attached to the bracket 84B. Therefore, when the belt driving part 800B drives the belt 89B, the bracket 84B and the three pulleys 85B, 86B, and 87B move together in the −x direction.

Furthermore, the rotating part 90 is axially supported by the brackets 84A and 84B. Therefore, when the belt driving parts 800A and 800B drive the belts 89A and 89B, respectively, the rotating part 90 also moves in the −x direction.

Furthermore, the brackets 84A and 84B are secured to the bottom surface of the structure 50. Furthermore, the driving device 70 has the two rails 83A and 83B, and the two rails 83A and 83B are also secured to the bottom surface of the structure 50. Therefore, when the brackets 84A and 84B move in the −x direction, the rail 83A slides in the −x direction in a condition supported by the linear bearings 81A and 82A, and the rail 83B slides in the −x direction in a condition supported by the linear bearings 81B and 82B. Therefore, as illustrated in FIG. 23, the structure 50 can be moved in the −x direction.

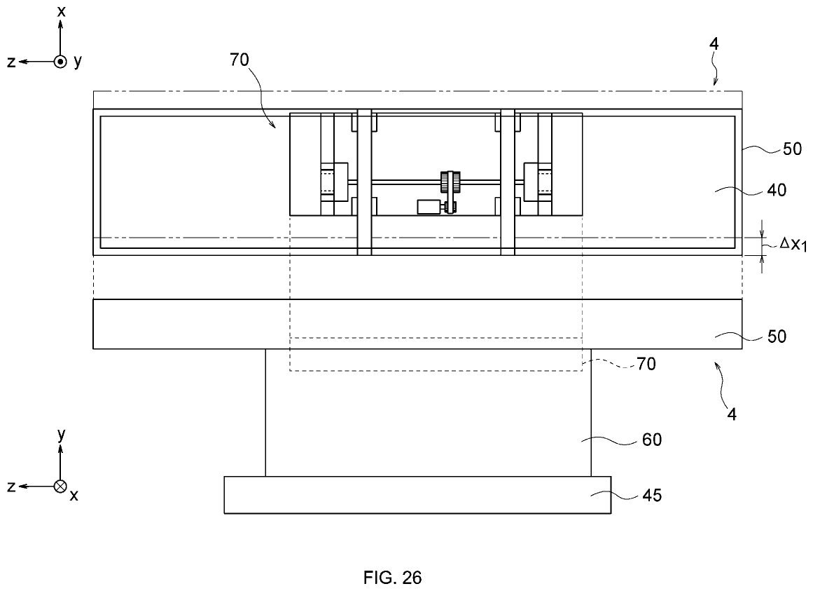

FIG. 26 is a plan view and side surface view of the table 4 when the structure 50 is moved in the −x direction. Note that in the plan view, the driving device 70 is illustrated by a solid line to make the structure of the driving device 70 easier to visually recognize. Furthermore, in the plan view, the structure 50 before being moved is illustrated by chain double-dashed lines, and the structure 50 after being moved is illustrated by a solid line. As illustrated in FIG. 26, the structure 50 can be moved in the −x direction by the predetermined distance Δx1.

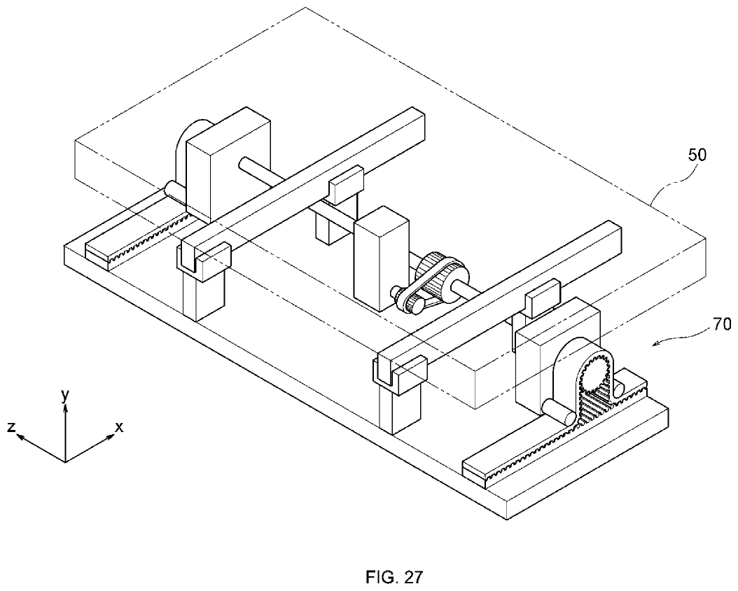

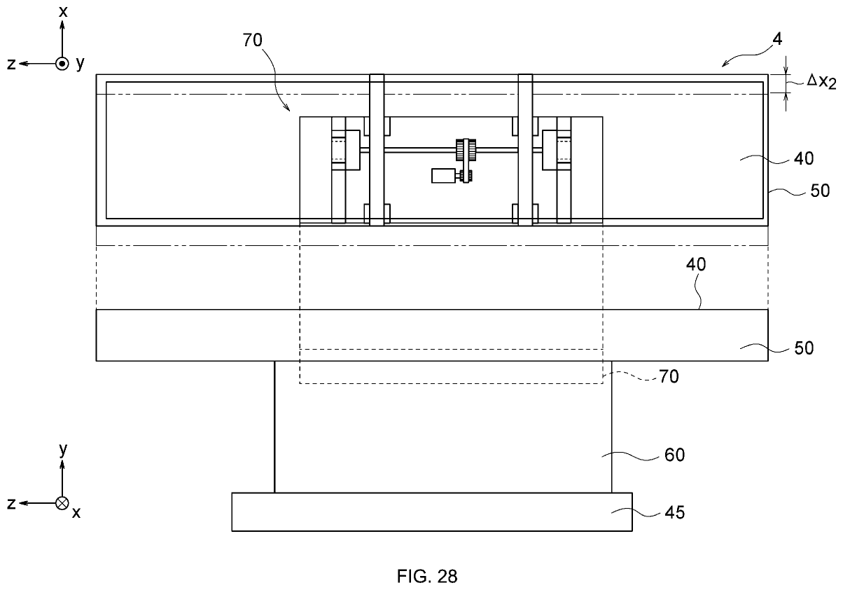

Movement of the structure 50 in the −x direction was described thus far, but by rotating the motor 92 in an opposite direction, the structure 50 can be moved in the x direction. FIG. 27 is a perspective view illustrating the structure 50 moved in the x direction by the driving device 70, and FIG. 28 is a plan view and side surface view of the table 4 when the structure 50 is moved in the x direction. As illustrated in FIG. 28, the structure 50 can be moved by Δx2 in the x direction.

As described above, in the present embodiment, the motor 92 rotates such that the structure 50 can move in the transverse direction. Therefore, even if it is difficult to set an imaging site of a patient in the x direction (transverse direction) to a suitable position for imaging by simply adjusting the posture of the patient, the imaging site of the patient can be set to an optimal position in the x direction by moving the structure 50 in the transverse direction.

Furthermore, in the present embodiment, the first conversion part 100A is provided on the end surface 72a side of the frame 71, and the second conversion part 100B is provided on the end surface 72b side of the frame 71. Therefore, the two conversion parts 100A and 100B provided separated in the z direction convert the rotational motion of the shaft 96 into a linear motion that moves the structure 50 in the transverse direction, and thus vibrations of the structure 50 in the yawing direction can be effectively reduced. Note that the shorter the interval between the belt driving parts 800A and 800B, the more likely that vibrations in the yawing direction will be generated. Therefore, the interval between the belt driving parts 800A and 800B is preferably increased in length as much as possible.

Furthermore, in the present embodiment, the pair of idler pulleys 86A and 87A press the belt 89A against the rack 88A while the belt driving part 800A is driving the belt 89A. Therefore, while the belt 89A is driven, the belt 89A firmly engages with the rack 88A, such that the movement of the belt 89A can be stabilized without the belt 89A coming off the rack 88A. Furthermore, the belt 89A firmly engages with the rack 88A, which reduces the effect of stretching of the belt 89A on the movement of the belt 8A. Similarly, the belt driving part 800B stably drives the belt 89B, which reduces the effect of stretching of the rail 83B.

In the present embodiment, the rail 83A is supported by the two bearings 81A and 82A, and the rail 83B is supported by the two bearings 81B and 82B. However, each rail can be supported by one or more bearings.

In the present embodiment, the structure 50 includes the cradle 40 and the cradle supporting part. However, the structure 50 may include only the cradle 40. The structure 50 is not required to include the entire cradle supporting part, and may include the cradle 40 and a part of the cradle supporting part. Furthermore, the structure 50 may include another component or member in addition to the cradle 40 and the cradle supporting part.

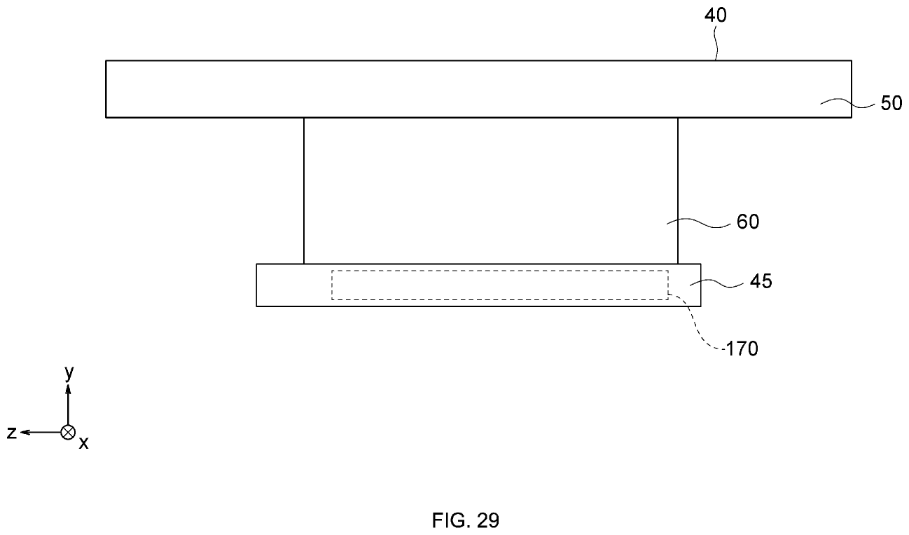

Furthermore, in the present embodiment, an example is indicated where the structure 50 moves in the transverse direction, but the present invention is not limited to examples where only the structure 50 moves in the transverse direction. For example, as illustrated in FIG. 29, a driving device 170 can be provided at a base 45 of the table 4 (refer to FIG. 3), and the driving device 170 can be secured to a bottom surface of the elevating part 60. By providing such a driving device 170, the structure 50 and the elevating part 60 can integrally move in the transverse direction.

What is claimed is:

1. A driving device that drives a table provided with a structure including a cradle, comprising:
a shaft rotatable about a rotation axis along a longitudinal direction of the cradle;
a first conversion part provided on a first end side of the shaft and that converts a rotational motion of the shaft into a linear motion for moving the structure in a transverse direction, where the transverse direction is a direction orthogonal to the longitudinal direction of the cradle and a height direction of the table; and
a second conversion part provided on a second end side of the shaft and that converts the rotational motion of the shaft into linear motion.

2. The driving device according to claim 1, wherein the first conversion part includes:
a first belt; and
a first belt driving part that drives the first belt, the first belt driving part drives the first belt to convert the rotational motion into the linear motion, the second conversion part includes:
a second belt; and
a second belt driving part that drives the second belt, and the second belt driving part drives the second belt to convert the rotational motion into the linear motion.

3. A driving device according to claim 2, wherein the first belt driving part includes:
a first rack that engages with the first belt;
a first bracket having a through hole through which the shaft passes;
a first drive pulley attached to a portion of the shaft protruding from the first bracket, which transmits the rotational motion of the shaft to the first belt; and
a first pair of idler pulleys that press the first belt against the first rack, where a first of the first pair of idler pulleys presses the first belt against the first rack between a first end portion of the first belt and a portion of the first belt that contacts the first drive pulley, and a second idler pulley presses the first belt against the first rack between a second end portion of the first belt and a portion of the first belt that contacts the first drive pulley, the first rack, the first drive pulley, and the first pair of idler pulleys maintain the first belt in a tensioned state, and when the rotational motion of the shaft is transmitted to the first belt by the first drive pulley, the rotational motion is converted to the linear motion.

4. The driving device according to claim 1, wherein while the first belt driving part drives the first belt, the first drive pulley, the first pair of idler pulleys, and the first rack tension the first belt, and the first pair of idler pulleys press the first belt against the first rack.

5. The driving device according to claim 4, wherein the first pair of idler pulleys are axially supported by the first bracket.

6. The driving device according to claim 3, wherein the first rack and the first belt are mated to each other.

7. A driving device according to claim 2, wherein the second belt driving part includes:
a second rack that engages with the second belt;
a second bracket having a through hole through which the shaft passes;
a second drive pulley attached to a portion of the shaft protruding from the second bracket, which transmits the rotational motion of the shaft to the second belt; and
a second pair of idler pulleys that press the second belt against the second rack, where a first of the second pair of idler pulleys presses the second belt against the second rack between a first end portion of the second belt and a portion of the second belt that contacts the second drive pulley, and a second idler pulley presses the second belt against the second rack between a second end portion of the second belt and a portion of the second belt that contacts the second drive pulley, the second rack, the second drive pulley, and the second pair of idler pulleys maintain the second belt in a tensioned state, and when the rotational motion of the shaft is transmitted to the second belt by the second drive pulley, the rotational motion is converted to the linear motion.

8. The driving device according to claim 7, wherein while the second belt driving part drives the second belt, the second drive pulley, the second pair of idler pulleys, and the second rack tension the second belt, and the second pair of idler pulleys press the second belt against the second rack.

9. The driving device according to claim 8, wherein the second pair of idler pulleys are axially supported against the second bracket.

10. The driving device according to claim 7, wherein the second rack and the second belt are mated to each other.

11. The driving device according to claim 1, further comprising:
a first rail secured to the structure and that extends in the transverse direction;
one or more first bearings that slidably support the first rail;
a second rail secured to the structure at a position separated from the first rail in a longitudinal direction of the shaft and that extends in the transverse direction; and
one or more second bearings that slidably support the second rail.

12. The driving device according to claim 1, wherein the structure includes the cradle and a cradle supporting part that supports the cradle.

13. A table that moves an imaging subject, comprising:
a structure includes a cradle that supports the imaging subject;
a shaft rotatable about a rotation axis along a longitudinal direction of the cradle;
a first conversion part provided on a first end side of the shaft and that converts a rotational motion of the shaft into a linear motion for moving the structure in a transverse direction, where the transverse direction is a direction orthogonal to the longitudinal direction of the cradle and a height direction of the table; and
a second conversion part provided on a second end side of the shaft and that converts the rotational motion of the shaft into a linear motion.

14. The table according to claim 13, wherein the structure includes the cradle and a cradle supporting part that supports the cradle.

15. The table according to claim 13, wherein the table includes an elevating part for adjusting the height of the table, and the first and second conversion parts convert the rotational motion of the shaft into a linear motion for moving the structure and the elevating part in a transverse direction.
